(19) United States
(12) Patent Application Publication
(10) Pub. No.: US 2011/0244466 A1
(43) Pub. Date: Oct. 6, 2011

(54) NUCLEIC ACID TESTING DEVICE AND METHOD

(76) Inventors: Robert Juncosa, Fairport, NY (US); Joel Grover, Pittsford, NY (US)

(21) Appl. No.: 13/078,282

(22) Filed: Apr. 1, 2011

Related U.S. Application Data
(60) Provisional application No. 61/320,553, filed on Apr. 2, 2010.

(51) Int. Cl. C12Q 1/68; C12M 1/10; C12M 1/34; C12P 19/34

(52) U.S. Cl. 435/6.12; 435/289.1; 435/287.2; 435/91.2

(57) ABSTRACT

Devices and systems for extracting, purifying and amplifying nucleic acids, and methods for use of such devices and systems. The devices have top sections which include a plurality of syringe vessels with applicable reagent materials, as well as a channel for a specimen collection dropper. Plungers force the materials sequentially into reaction chambers in the middle sections. Once the samples are extracted and purified, they are drawn by a vacuum into PCR devices where they are subjected to denaturing, annealing and extension steps and amplified. Interrogation of flow cells provide real-time quantitative detection.

### CROSS REFERENCE TO RELATED APPLICATION

The present invention claims priority to U.S. provisional application 61/320,553, filed Apr. 2, 2010, the disclosure of which is incorporated herein by reference.

### TECHNICAL FIELD

This invention is directed to systems and methods of extracting. purifying and amplifying DNA and other nucleic acids, and more particularly to devices for accomplishing this.

### BACKGROUND

Deoxyribonucleic acid (“DNA”) is a nucleic acid that contains the genetic instructions used in the development and functioning of virtually all known living organisms. The main role of DNA molecules is the long-term storage of information. DNA contains the instructions needed to construct other components of cells, such as proteins and RNA molecules. Within cells, DNA is organized into long structures called chromosomes, and the genes are the DNA segments that carry the genetic information.

The polymerase chain reaction (“PCR”) is a scientific technique in molecular biology to amplify a single or a few copies of a piece of DNA across several orders of magnitude, generating thousands or more copies of a particular DNA sequence. The method relies on thermal cycling, consisting of cycles of repeated heating and cooling of the reaction for DNA melting and enzymatic replication of the DNA.

PCR is a technique used in medical and biological research for a variety of applications. These include the diagnosis of hereditary diseases, the identification of genetic fingerprints (used in forensic sciences and paternity testing), the detection and diagnosis of infectious diseases, and DNA cloning for sequencing, DNA-based phylogeny, or functional analysis of genes. PCR permits the early diagnosis of malignant diseases such as leukemia and lymphomas. PCR also permits identification of non-cultivatable or slow-growing microorganisms such as mycobacteria, anaerobic bacteria, or viruses from tissue culture assays and animal models.

Methods and systems for extracting nucleic acids, DNA or RNA, and performing PCR techniques today for the most part suffer from one or more defects, such as being too expensive, too time-consuming to perform, and/or too complex and difficult to use. The manipulation of the sample for testing generally requires a skilled technician or scientist and is not usually “automation friendly.” A typical format for PCR is in trays containing open wells which also require significant manual interaction by skilled operators and are subject to contamination.

There is a need today for a system and method which combines DNA extraction and PCR amplification and detection in a faster, less costly and easier manner, and requires little or no operator intervention.

### SUMMARY OF THE INVENTION

The present invention provides devices, systems and methods for accomplishing and meeting these objectives. The invention provides integrated devices which can perform the steps necessary for a nucleic acid test, including nucleic acid extraction, purification and reverse transcription (RT) in the case of RNA, PCR amplification, and real time detection. Extraction, purification and RT, if required, are often referred to as sample preparation. The devices can be single use disposable devices which are relatively small, inexpensive, and easy to manufacture and assemble.

The devices can be used manually, or the devices can be operated by a machine which performs the steps automatically.

A preferred embodiment of a device has three sections: an upper or top section, a middle or center section, and a lower or bottom section. The top section stores reagents for sample preparation and purification. The collected specimen to be tested is also stored in the top section.

The middle section holds a reaction chamber. This is where sample preparation takes place. Ports are provided for connection to a pneumatic or hydraulic system to enable movement of fluids through the device. The middle section also contains areas for collection of waste.

The lower section provides PCR amplification and detection. Preferred PCR devices for this purpose are disclosed and described in U.S. Pat. No. 7,618,811 and U.S. patent application Ser. No. 11/744,676.

A preferred method of the invention involves use of a preferred device to perform nucleic acid extraction and purification, PCR amplification, and detection.

Other objects, features, benefits and advantages of the present application will become apparent from the following description of preferred embodiments of the invention when viewed in accordance with the accompanying drawings and appended claims.

### BRIEF DESCRIPTION OF THE DRAWINGS

FIG. 1 schematically depicts a preferred embodiment of an inventive device for performing the inventive method.

FIG. 2 is an exploded view of the device illustrated in FIG. 1.

FIG. 3 is an exploded view of the top section of the device of FIG. 1, together with a collection dropper member.

FIG. 4 depicts the middle section of the device shown in FIG. 1.

FIG. 5 is a cross section of the middle section shown in FIG. 4, the cross section taken along lines 5-5 in FIG. 4.

FIG. 6 is an exploded view of the middle section of the device of FIG. 1.

FIG. 7 schematically depicts a PCR amplification device (in exploded view) which can be used as part of an embodiment of the present invention.

FIG. 8 schematically illustrates PCR cycling channels in the device shown in FIG. 7.

FIGS. 9 and 10 illustrate an initial step in the use of the device shown in FIG. 1.

FIGS. 11 and 12 illustrate a subsequent step in the use of the device shown in FIG. 1.

FIGS. 13-18 depict additional steps in the use of the device shown in FIG. 1, with FIG. 16 illustrating magnetic beads.

FIG. 19 schematically depicts detection of the nucleic acid in a PCR amplification device.

### DESCRIPTION OF THE PREFERRED EMBODIMENTS

For the purpose of promoting and understanding the principles of the present invention, reference will now be made to the embodiments illustrated in the drawings and specific language will be used to describe them. It will nevertheless be understood that no limitation as to the scope of the invention is hereby intended. The invention includes any alternatives and other modifications in the illustrated devices and described methods and further applications of the principles of the invention which would normally occur to persons or ordinary skill in the art to which the invention relates.

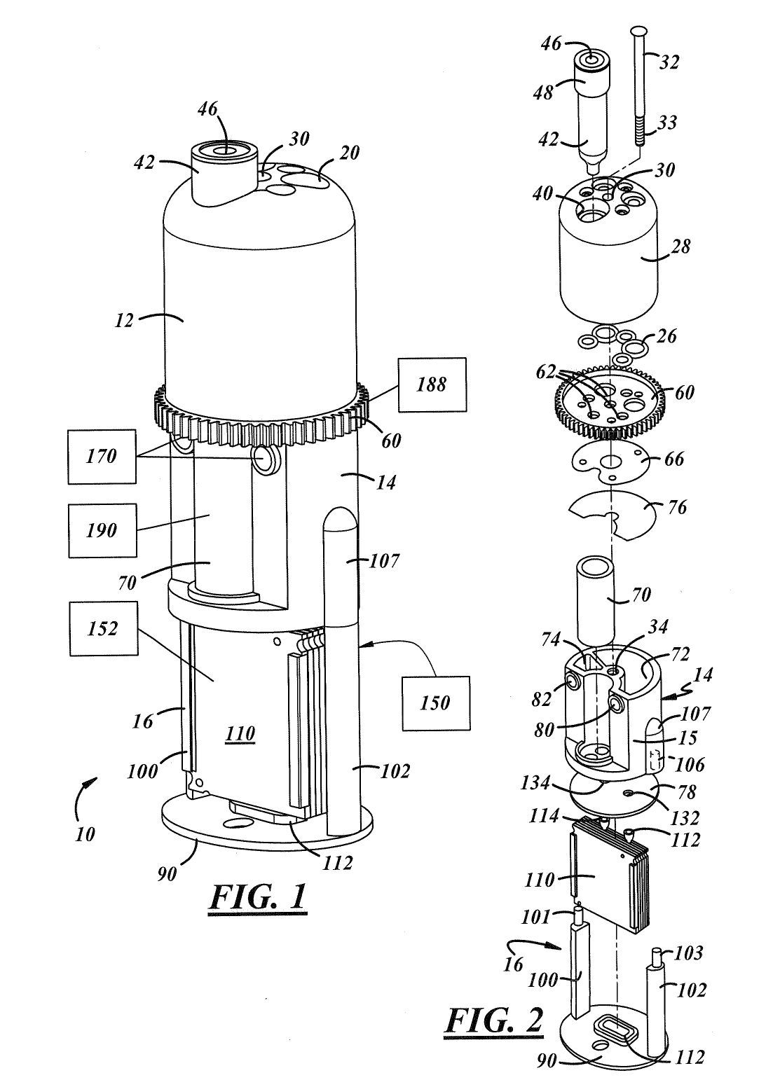

A preferred embodiment of a device in accordance with the present invention is shown in FIG. 1 and designated generally by the reference number 10. An exploded view of that device 10 is shown in FIG. 2.

The device 10 has three sections: a top or upper section 12, a center or middle section 14, and a bottom or lower section 16. In general, the top section 12 holds and stores reagents for sample extraction and purification.

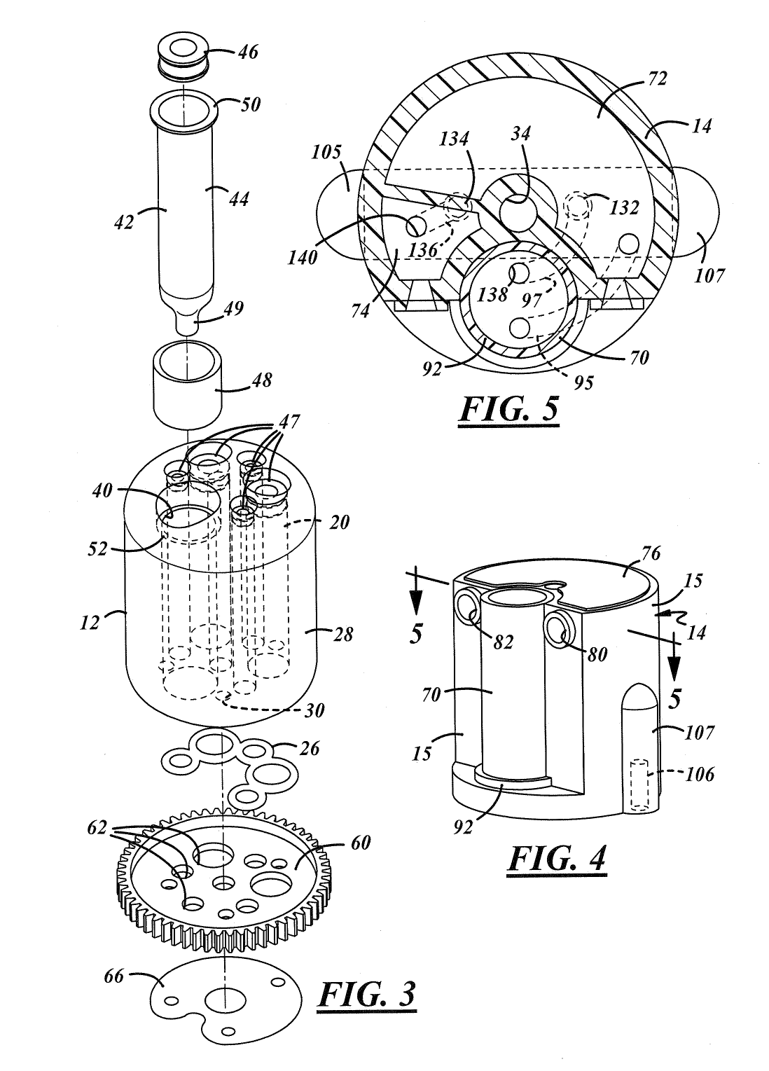

As shown in FIG. 3, there are a plurality of syringe tubes 20 positioned in the housing 28 in the top section for storing the reagents. In the device depicted, there are five syringe tubes, although the number of tubes and their contained volume capacity can be optimally designed depending on the precise nucleic acid being tested and the desired method of extraction and purification.

Each of the syringe tubes 20 is filled with a different reagent, which can be a liquid, a suspended solid, or a combination thereof The diameter of the syringe tube—and thus its volume—is selected according to the amount of the particular reagent needed for the desired process.

The syringe tube members can be plastic or glass syringe bodies with tapered outlet ends positioned in cylindrical channels in the housing 28. The channels in the housing could also be formed in the shape of syringe tubes with tapered lower ends, or simply be elongated channels with constant cross-sections. If syringe tube members are utilized, they preferably have flanges on their upper ends which are supported on the top surface of the housing.

A stopper member 47, preferably made from a rubber or elastomeric material, is inserted in the upper end of each reagent syringe tube member in order to seal in the reagent and keep it from being contaminated. Similar to injection syringes, the stopper members 47 are designed to be slid down the insides of the tubes 20 when a force is applied in order to dispense reagents from the other ends. The lower ends of the syringe tubes may be tapered to assist in dispensing the reagents from the tubes.

Seal members 26 are provided at the lower ends of each of the syringe reagent tubes 20. The seals can be individual seals for each tube, or, as shown in FIGS. 2 and 3, can be provided in a group of collective seals for ease of manufacture and assembly. The seals can be preferably made from a material with a consistent tensile failure limit so that the seals burst predictably. One such material is polypropylene. Alternatively, seal members may be septums or check valves that open at a predictable pressure. These approaches have the advantage of allowing a portion of the reagent to be dispensed repeatedly from the same silo.

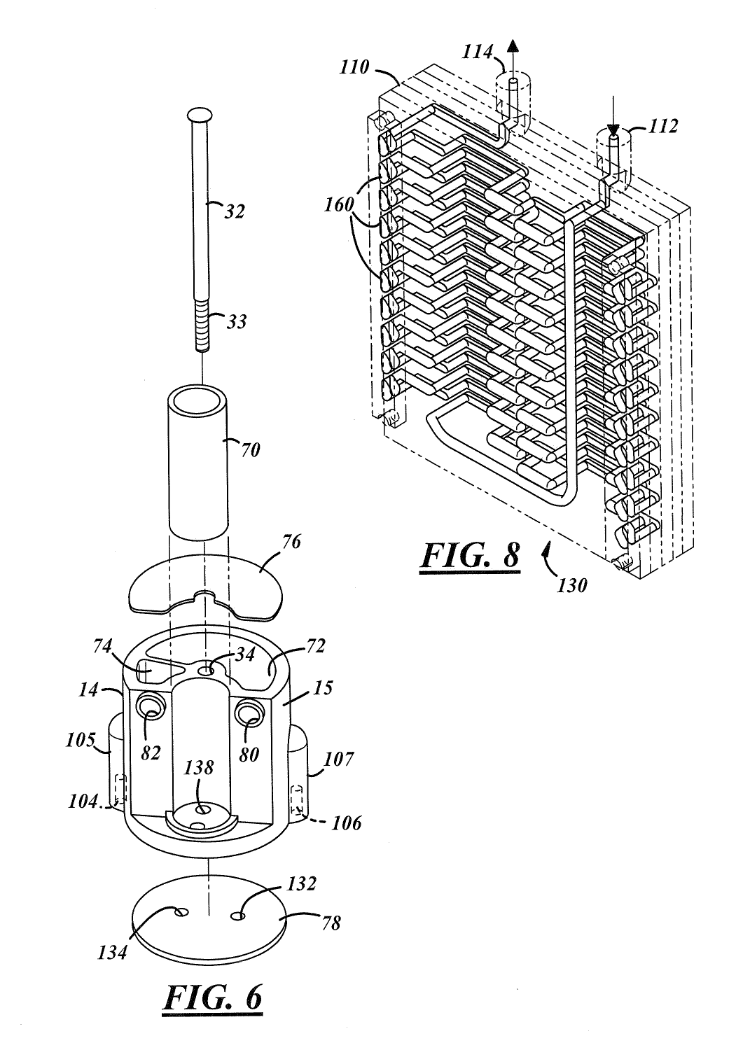

The housing member 28 has a central opening 30 in which a pin 32 is positioned. The pin 32 is used to hold the top section 12 to the middle section 14. As shown in the drawings, particularly FIGS. 2, 3 and 6, the pin 32 passes through the top section and other components and is secured in central channel 34 in the middle section 14. The pin 32 may have a threaded lower end 33 so it can be threadedly connected into the central channel 34 to securely hold the two sections 12 and 14 together. Alternatively, the pin may mate and be secured through the use of a “press fit” frictional process, or with a glue or another adhesive. The pin acts as a pivot pin to allow the housing 28 to rotate relative to the middle section.

The top section 12 also has a cylindrical channel 40 for placement and holding of a specimen collection dropper member 42. As shown in FIG. 3, dropper member 42 has a pliable body member 44, a stopper member 46, and a tapered lower end 49. The dropper, when squeezed, is used to collect a specimen. The sample can be a bodily fluid, such as blood, or a specimen of cell tissue. In the case of a blood specimen, such an extraction may be made directly from a finger, earlobe or heel prick.

Once the sample is collected, the collection dropper member 42 is inserted in channel 40. In a preferred embodiment, a ring member 48 is also provided to insure that the dropper 42 is located in the desired position in the channel 40 in the top section. The ring member 48 is positioned around the body member 44 and seated at the one end under rim flange 50 on the body member and upon stop 52 formed in the channel 40.

The stopper member 46 is inserted in the upper end of the body member 44 and is designed to slide down the inside of the body member when a force is applied to it in order to dispense the collected sample through the lower end 49.

In an automated process, the specimen collection dropper 42 can remain with the top section during the further steps in the process. This minimizes further handling by an operator and reduces opportunities for contamination.

As an alternate embodiment, a simple capillary tube could be used to collect the blood sample and dispense it into the device.

Also included as part of the top section 12 is a gear wheel 60. The gear wheel is secured to the housing member 28 and, as described below, is used to rotate the top section 12 relative to the middle section 14 when the steps of the process are being performed. The gear wheel 60 has a plurality of openings 62 which correspond to and are aligned with the syringe reagent tube members 20 and collection dropper opening 40 in the housing. The openings in the gear wheel also are aligned with the seal members 26 and hold the seals tightly against the bottom of the housing 28. This helps prevent leakage of the reagents, and also assists in the opening of the seal members (as described below) in the steps of the extraction and purification processes.

A mesh or screen member 66 is also provided, and is positioned on the side of the gear wheel 60 opposite the seal members 26. The screen member prevents any loose pieces of seal members after they are opened from passing through into the middle section 14. The screen also can have solid reagents coated on it (immobilized) so that they (i.e. the solid reagents) can be released by a liquid which is dispensed from one or more of the syringe reagent tubes. Magnetic beads could also be deposited or immobilized (attached) on the screen so they can be dislodged and dispensed along with the liquid from one of the reagent tubes. The magnetic beads can also be introduced into the reaction chamber in various other ways known to persons of ordinary skill in the art.

The middle section 14 of the device 10 includes a housing 15, the reaction chamber 70, two waste reservoir chambers 72 and 74, and two pneumatic vacuum ports 80 and 82. A cover member 76 is positioned on the housing 15 to cover the two waste chambers. A plate member 78 encloses the lower end of the housing 15.

The reaction chamber 70 is made of a pliable material and is tightly positioned in well member 92. A pair of outlet openings 94 and 96 are located in the well member 92 and are in communication with passageways 95 and 97 for reasons explained below. The outlet openings 94 and 96 are sufficiently small such that, because of capillary pressure, the reaction chamber will not drain due to gravity or mechanical disturbance of either the device 10 or reaction chamber 70.

The bottom or lower section 16 has a base member 90 which allows the device to be positioned upright as shown in the drawings. A locating or attachment opening 92 in the base member 90 allows the device 10 to be accurately positioned in an automated mechanism (if the device 10 is being operated automatically), or anchored, if desired, to a table or work platform for manual operation.

The lower section is attached to the middle section by a pair of upright support members 100 and 102. The support members are affixed to the base member 90 and have pin members 101 and 103 that fit in mating sockets 104 and 106 in side cap members 105 and 107 in the housing member 15.

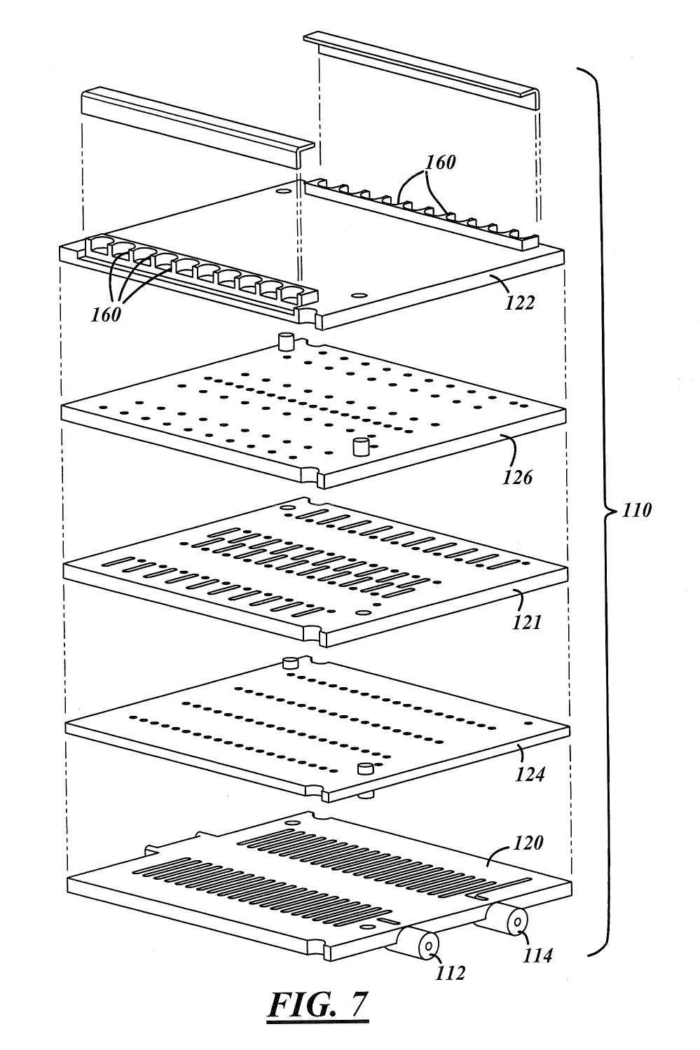

A PCR amplification device 110 is positioned in the lower section. It is positioned on a platform 112 on the base member 90. The PCR device 110 is preferably a real-time PCR thermal cycling device as disclosed in U.S. Pat. No. 7,618,811, or U.S. patent application Ser. No. 11/744,676, filed May 4, 2007, the disclosures of which are hereby incorporated by reference herein. The PCR device 110 can be either a two or three layer device; the representative device shown and described herein is a three-layer embodiment, as shown in more detail in FIGS. 7-8. The three thermally conductive layers are numbers 120, 121 and 122 and are separated by two insulating layers 124 and 126.

It is to be understood that the PCR device can also be any one of the other embodiments disclosed or claimed in U.S. Pat. No. 7,618,811, or U.S. patent application Ser. No. 11/744,676, whether two or three layers, and whether or not with insulating layers.

The PCR device 110 also has an inlet port 112 and an outlet port 114. When the nucleic acid sample is dispensed into the port 112, it is passed through a channel structure 130, such as that depicted schematically in FIG. 8. The segments of the channels are positioned in all three layers so that the steps of denaturation, annealing and extension can be performed repeatedly on the sample.

When the middle section 14 and lower section 16 are assembled together with the PCR device 110, the ports 112 and 114 are positioned in, or are in sealing engagement with, openings 132 and 134 in plate member 78. Opening 132 is in communication through passageway 97, with opening 138 in the reaction chamber well member 92. Opening 134 is in communication through passageway 136 with opening 140 in waste chamber 74. In this manner, when a specimen to be amplified has been extracted and purified in reaction chamber 70, it is pulled by a vacuum through opening 138, through passageway 97, through opening 132 and into the PCR device 110 through inlet port 112. Then, after the amplification of the sample is completed by being subjected to thermal cycling, it exits the PCR device through outlet port 114. The sample then passes through passageway 136, and enters the waste chamber 74 through opening 140. Since the device 10 and PCR device 110 are preferably disposable, the remains of the sample in waste chamber 74 are simply disposed along with the device 10.

In the PCR device 110, the sample is first exposed to a long denaturing step. This is performed in the first layer 120 of the device 110. The sample then moves to the rear (third) layer 122 and is subjected to an annealing step. After annealing, the sample moves to the middle layer for extension, thus completing one thermal cycle. The sample is then routed back to the front layer for the denaturing step where it completes a second thermal cycle. This process continues through the length of the PCR device until the sample exits. This is described in more detail in U.S. Pat. No. 7,618,811 and U.S. patent application Ser. No. 11/744,676.

In the denaturing step, the sample is heated to about 94-98° C. The first thermally conductive layer 120 of the PCR device is heated by a heating device 150 which is placed in contact with that side of the PCR device. This is shown schematically in FIG. 1. In the annealing step, the temperature of the sample is lowered to about 50-65° C. The third thermally conductive layer 122 of the PCR device is kept at that temperature by a heating device 152 which is placed in contact with the other side of the PCR device. In the extension step, the middle layer 121 of the PCR device is heated to about 75-80° C. The middle layer 121 does not have to be heated by a separate heating source, but can assume its temperature due to its location between layers 120 and 122. It is possible, however, to also have a third heating source for the middle thermally conducting layer.

The heating sources 150 and 152 can be any conventional type of heating sources which can be used to place the layers 120 and 122 in their desired temperatures. Heat sources which can be used with this embodiment of the present invention include direct contact mechanisms, such as heating elements, or indirect mechanisms such as inductive, acoustic, or radiation heating.

The various components of the device 10 are preferably made of a molded plastic material, such as polypropylene, polycarbon or polyetherimide. These materials are relatively inexpensive and can be easily molded. They also can be trimmed, drilled and finished relatively easily in order to provide the desired components. Preferably, the device 10 has an overall size on the order of 1-1½ inches in diameter by 4-5 inches in height.

After each extension step in the PCR device 110, the sample can be routed to a flow cell 160 mounted on the denaturing layer of the device. The flow cells have clear windows which allow interrogation by a laser light or the like. The amount of amplification of the nucleic sample can be detected in real time by fluorescent detection. This is represented by wavy lines 162 in FIG. 19. The PCR sample mix, as it progresses through the PCR device, becomes more fluorescent with each cycle.

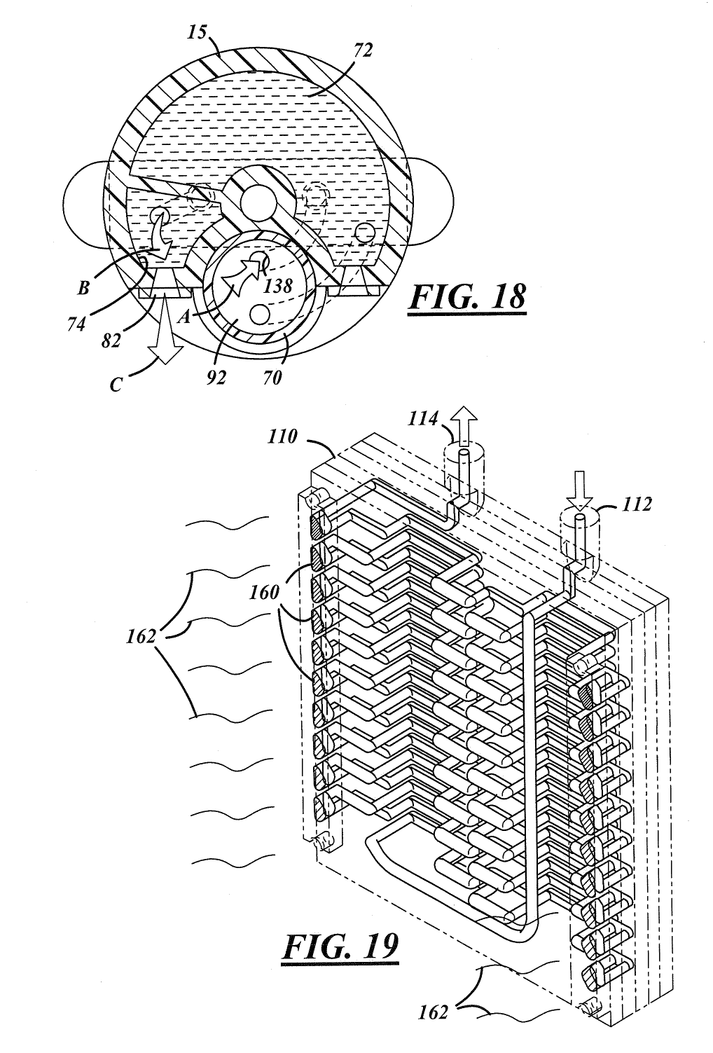

Movement of the sample from the reaction chamber and through the PCR device and finally into the waste reservoir chamber 74 is accomplished by a vacuum mechanism or system 170. A vacuum system of any conventional type is connected to pneumatic ports 80 and 82 in the housing 15 in the middle section 14. By pulling a vacuum at the port 82, a vacuum is created through the waste chamber 74, through the cycling channel 130 in the PCR device and through the opening 138 in the well 92 at the bottom of the reaction chamber 70. This is schematically illustrated in FIG. 18 where the sample being pulled from the well is indicated by arrow A and the amplified sample entering the waste chamber 74 is indicated by arrow B. The vacuum used in this processing step is indicated by the arrow C in FIG. 18.

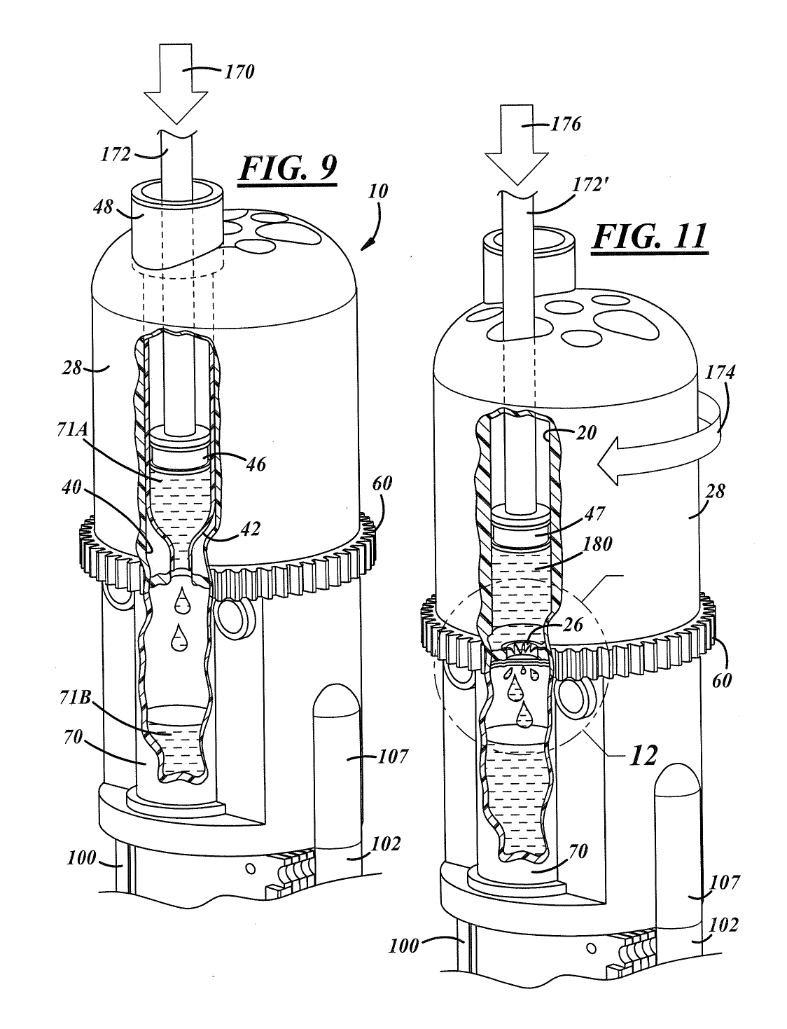

The first step in the testing process is to collect the specimen and introduce it into the reaction chamber 70. Once the specimen is collected as indicated above, the collection dropper 42 is positioned in the opening 40 in the housing 28. Of course, it is also necessary to first make sure that the opening 40 is aligned with the reaction vessel 70 as shown in FIG. 9. The stopper member 46 is then forced downwardly in the dropper tubular member forcing the specimen into the reaction vessel. This step is shown schematically in FIG. 9.

The specimen, which is shown partially inserted in FIG. 9, is identified by the numerals 71A and 71B. When a force represented by arrow 170 acts on a plunger member 172 in the manner shown, the specimen 71A in the dropper 42 is injected (dispensed) into the reaction chamber 70 and is represented as 71B.

The plunger 172 can be forced downwardly in the collection dropper member manually, or automatically by a processing mechanism for the device 10.

Once the specimen is fully injected into the reaction chamber 70, the housing 28 is rotated again, either manually or preferably automatically by a processing mechanism 188, to position another syringe reagent tube over and in alignment with the reaction chamber. The rotation of the housing is accomplished by rotation of the gear wheel 60.

The specimen dropper can be removed and discarded at this point, or at any other point in the subsequent process. It is no longer needed. Preferably, however, the specimen dropper is allowed to remain with the device 10, thereby reducing opportunities for contamination.

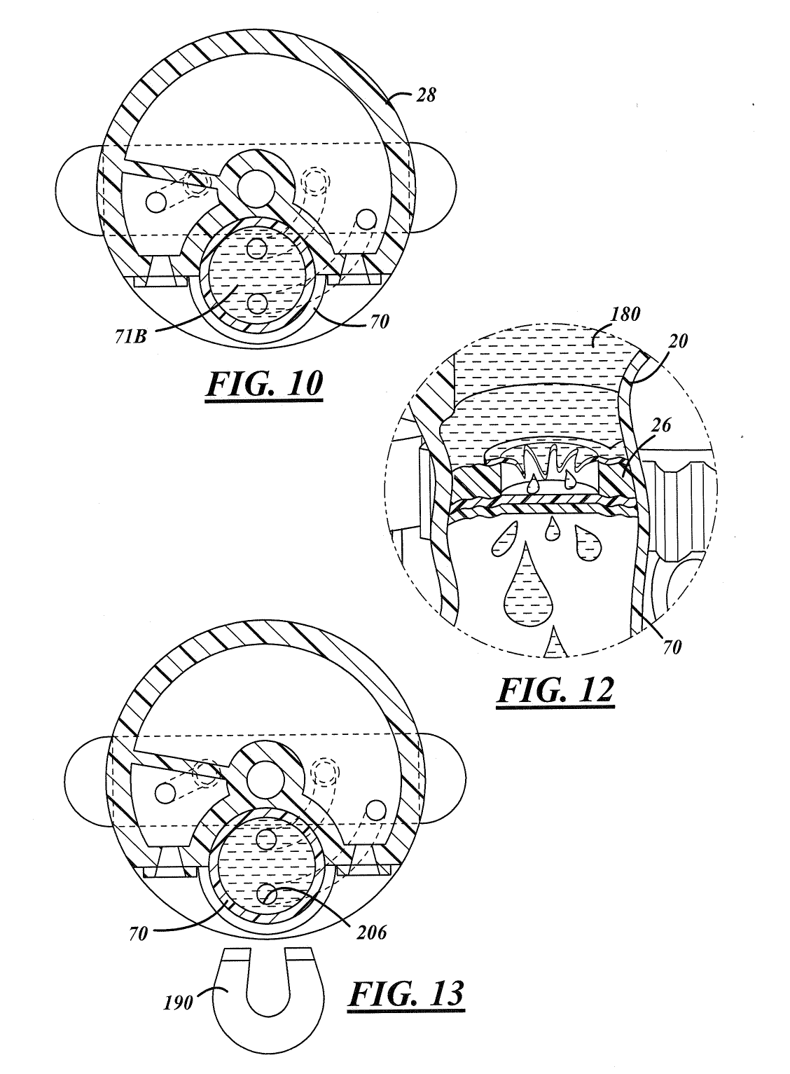

FIG. 10 is a cross-section of the housing 28 and reaction vessel 70 showing the specimen 71B in the reaction vessel and ready for future processing.

Once the specimen has been introduced into the reaction vessel 70, subsequent reagents, either liquid, solid, or both, are sequentially dispersed into the reaction vessel 70 in a similar manner. The housing 28 is rotated as represented by arrow 174 in FIG. 11 until the next appropriate syringe reagent tube is positioned above the reaction chamber 70.

The amount and type of reagents in the syringe tubes, and the sequencing of the injection of the reagents from the syringe tubes depends on the specific protocol required to perform the test, the type of specimen, such as blood, urine, cerebrospinal fluid, tissue, or sputum, and the type of nucleic acid to be extracted, purified and, if required, reverse transcribed, such as viral RNA, bacterial DNA, genomic DNA, messenger RNA.

Also, typical reagents used for PCR processes which can be used in the device 10 include cell lysing agents, protenases, washing and elution buffers. Other possible reagents that can be utilized in the syringe tubes are nucleic acid triphosphates, primer and probe oligonucleotides, DNA polymerase and reverse transcription enzymes and buffering salts and/or solutions.

Nucleic acid, DNA or RNA, lies within living cells or viruses and is protected by various coatings or walls that must be dissolved with strong reagents to release them into solution. The debris (or waste) of this process are proteins which are undesirable in the amplification step. To remove it, the nucleic acid is first captured on a solid surface such as silica and then washed with one or more solutions. The purified nucleic acid is eluted with a buffered solution.

Once each subsequent syringe reagent tube is positioned over the reaction vessel, a plunger 172′, which preferably is the same plunger 172 used to inject the specimen and other reagents, is used to dispense the next reagent into the reaction chamber 70. As the plunger 172′ is forced downward in the direction of arrow 176, the stopper member 47 used to seal the end of the syringe tube 20 is also forced downwardly. This causes reagent liquid 180 to burst through the seal member 26, as shown schematically in FIGS. 11 and 12. The increased pressure in the syringe tube can cause the seal to open. The seal also could have a small slit preformed in it which stays closed due to natural compression caused by the weight of the reagent. Once the increased pressure from the plunger exceeds the slit seal holding capability, the liquid is dispensed from the tube.

Once a reagent is dispersed into the reaction chamber and reacts with the specimen, the waste material is removed and another syringe tube is rotated over the reaction vessel in order to perform the next step in the process. The stopper member and plunger act to seal the syringe reagent tube after the reagent is delivered to the reaction chamber 70. This process continues step-by-step, syringe by syringe, until the desired reactions are complete and the nucleic acid is extracted and purified.

If mixing of the materials in the reaction chamber are required, an agitation or mixing mechanism is utilized, such as a conventional sonicating, shaking or vibrating mechanism. The mechanism (not shown) can be positioned adjacent to or in contact with one of the walls of the reaction chamber 70.

In order to capture the nucleic acid in the reaction chamber through the reagent processing steps, magnetic beads are preferably utilized. These are beads which are specifically designed for processes such as PCR processes. The beads are microscopic and are typically coated with a material such as silica in order to bind the nucleic acid to them.

The magnetic beads can be inserted into the reaction chamber in several ways. For example, they can be provided in the reaction vessel initially, they can be attached to the mesh filter 66 by agents that dissolve when one of the reagents is dispensed, carrying them into the reaction chamber, or they can be included in one of the liquid reagents.

Once the reaction with each reagent is complete and it is necessary to eliminate the waste reagent material, a magnetic mechanism is used to hold the magnetic beads in the reaction vessel and prevent them from being exhausted with the waste products. This is shown schematically in FIG. 13. The magnet 190, which can be an electromagnet, is positioned immediately adjacent one or more walls of the reaction chamber 70 for this purpose. The magnet 190 can be activated manually, or preferably as part of an automated mechanism or machine used to perform the entire test procedure with the device 10.

Activation of the magnet 190 attracts and captures the magnetic beads 200 with the nucleic acid on them to the side of the reaction vessel 70, as shown in FIGS. 15 and 16. This allows the waste materials to be exhausted from the reaction vessel without exhausting the key part of the sample. The exhausted waste materials are shown as 202 in waste reservoir 72 in housing 15.

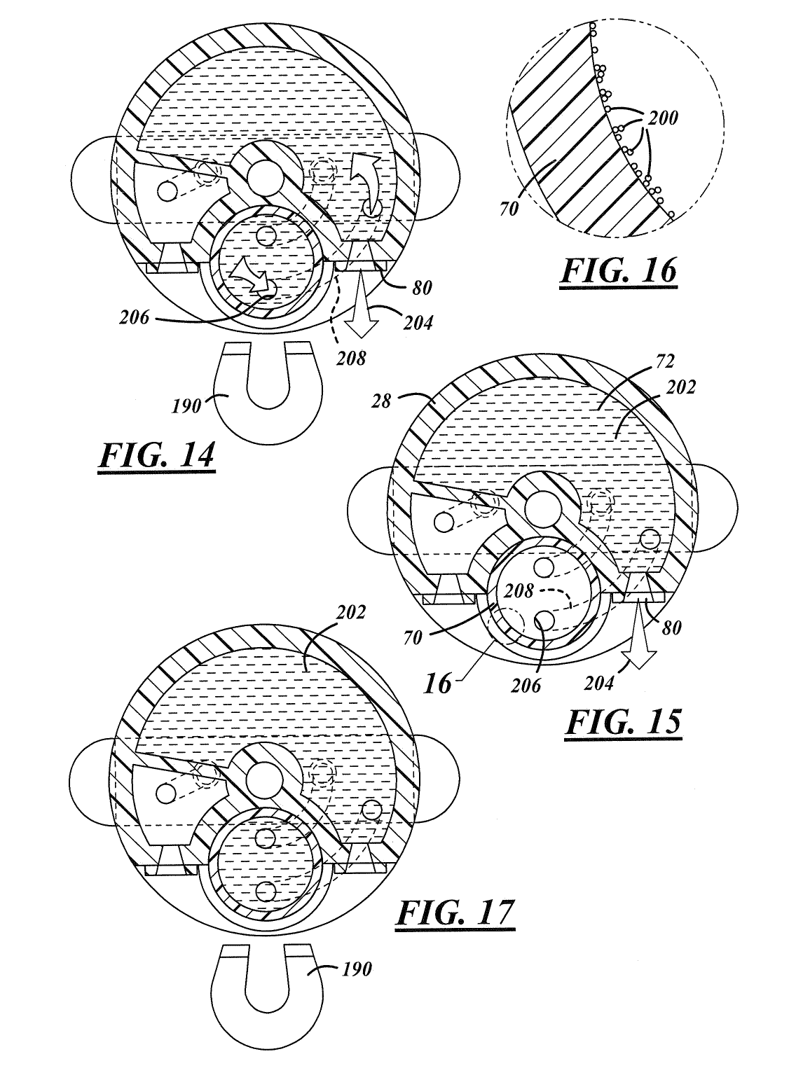

In order to remove the waste materials 202 from the reaction chamber 70, vacuum mechanism 170 is attached to pneumatic port 80, as shown in FIGS. 14 and 15. The vacuum mechanism is represented by numeral 170 in FIG. 1 and can be any conventional vacuum source that has the strength and ability to accomplish the tasks necessary in accordance with the inventive process. The vacuum source 170 pulls a vacuum through port 80 in the direction of arrow 204. The port 82 opens up directly into waste reservoir 72 which is in communication with opening 206 in well 92 through passageway 208.

Once the nucleic acid sample has been extracted with various reagents and has been washed and purified, the nucleic acid is defracted from the magnetic beads. This is also called lysis. Then, with the magnetic beads captured magnetically in order to remain in the reaction chamber, the vacuum is applied to pneumatic port 80 and the remaining solution with the extracted nucleic acid sample is pulled into the PCR device 110 for amplification. FIG. 17 illustrates the step immediately prior to initiating the vacuum through port 80.

Once the sample preparation process is complete and the purified sample is contained in the reaction chamber 70, the system draws a vacuum from pneumatic port 82 and the sample is drawn into the inlet 112 of the PCR device 110 located below the reaction vessel. As indicated above, once the sample moves through the entire PCR device and amplification is complete, it exits and is drawn into the small waste reservoir 74. At this point, the test is complete and the entire device 10 can be disposed of.

Although the invention has been described with respect to preferred embodiments, it is to be also understood that it is not to be so limited since changes and modifications can be made therein which are within the full scope of this invention as detailed by the following claims.

What is claimed is:

1. A device for extracting and purifying nucleic acids from a specimen, said device comprising:
a rotatable housing member having a plurality of reagent vessels for storing reagents;
a reaction chamber positioned to collect reagents dispensed from said reagent vessels and for conducting reactions therein;
a first waste reservoir for collecting waste materials from reacting in said reaction chamber.

2. The device as described in claim 1 further comprising a PCR device for amplifying materials after having been subjected to reactions in said reaction chamber.

3. The device as described in claim 1 further comprising a collection dropper member for collecting a sample to be dispensed into said reaction chamber.

4. The device as described in claim 3 further comprising a channel in said rotatable housing for insertion and storage of said collection dropper member.

5. The device as described in claim 1 further comprising a second housing member, said reaction chamber and said first waste reservoir being positioned on said second housing member.

6. The device as described in claim 5 further comprising a PCR device and a second waste reservoir in said second housing member, said second waste reservoir for collecting waste material from said PCR device.

7. The device as described in claim 5 further comprising a pin member rotatably securing said rotatable housing member to said second housing member.

8. The device as described in claim 1 further comprising a base member for holding said device in an upright manner.

9. The device as described in claim 1 further comprising a gear wheel on said rotatable housing member for selectively rotating said rotatable housing member.

10. A system for extracting, purifying and amplifying nucleic acids from a specimen, said system comprising:
a device having an upper portion, a middle portion, and a lower portion;
said upper portion comprising a first rotatable housing member having a plurality of reagent vessels for storing reagents, a channel for storage of a collection dropper member, a collection dropper member, and a gear wheel for rotating said upper portion;
said middle portion comprising a second housing member having a reaction chamber, two pneumatic ports, a first waste reservoir and a second waste reservoir;
said lower portion comprising a base member and a PCR device;
at least one heat conducting mechanism for applying heat to said PCR device; and
a vacuum mechanism for applying a vacuum to said two pneumatic ports.

11. The system as described in claim 10 further comprising a magnetic mechanism positioned adjacent said reaction vessel.

12. The system as described in claim 11 wherein said magnetic mechanism is an electromagnetic mechanism.

13. The system as described in claim 10 further comprising a detection mechanism for detecting amplification of nucleic acids in said PCR device.

14. The system as described in claim 13 wherein said detection mechanism is a fluorescent mechanism.

15. The system as described in claim 10 wherein said reagent vessels comprise a syringe tube, a stopper member and a seal member.

16. The system as described in claim 10 further comprising a collective seal member sealing positioned on the lower ends of each of said reagent vessels.

17. The system as described in claim 10 further comprising a screen member positioned on said first rotatable housing member adjacent the lower ends of said reagent vessels.

18. The system as described in claim 17 wherein said screen member has solid reagent materials immobilized thereon.

19. The system as described in claim 17 wherein said mesh filter member has micro magnetic silica beads immobilized thereon.

20. The system as described in claim 10 wherein said collection dropper member has a stopper member and a ring member.

21. The system as described in claim 10 wherein said collection dropper member comprises a pliable tubular member.

22. The system as described in claim 10 wherein said reaction chamber has two outlet ports, a first port in communication with said first waste reservoir and a second port in communication with said PCR device and said second waste reservoir.

23. The system as described in claim 10 wherein the first of said two pneumatic ports is in communication with said first waste reservoir and the second of said two pneumatic ports is in communication with said second waste reservoir.

24. The system as described in claim 10 wherein said upper portion and said middle portion are rotatably connected together by a pin member.

25. The system as described in claim 10 further comprising a pair of post members on said base member of said lower portion, said pair of post members connecting said lower portion to said middle portion.

26. The system as described in claim 22 wherein said two outlet ports are of a size to prevent flow of a liquid therethrough by gravity.

27. The system as described in claim 10 wherein said specimen is extracted and purified in said reaction vessel, and said specimen is subsequently amplified in said PCR device.

28. A device for extracting and purifying nucleic acids from a specimen, said device comprising:
a rotatable housing member having a plurality of reagent vessels for storing reagents;
a reaction chamber positioned to collect reagents dispensed from said reagent vessels and for conducting reactions therein;
a first waste reservoir for collecting waste materials from reacting in said reaction chamber; and
a second housing member, said reaction chamber and said first waste reservoir being positioned on said second housing member.

29. The system as described in claim 28 further comprising a PCR device and a second waste reservoir in said second housing member, said second waste reservoir for collecting waste material from said PCR device.

30. A method of extracting and purifying a nucleic acid sample, said method comprising the steps of:
collecting a specimen;
inserting said specimen into a reaction chamber in a testing device, said testing device comprising:
a rotatable housing member having a plurality of reagent vessels for storing reagents;
a reaction chamber positioned to collect reagents dispensed from said reagent vessels and for conducting reactions therein;
a first waste reservoir for collecting waste materials after reacting in said reaction chamber;
sequentially subjecting said specimen in said reaction chamber to reagents from said reagent vessels; and
exhausting waste reaction materials in said reaction chamber to said first waste reservoir after each sequential reagent reaction;
wherein an extracted and purified nucleic acid sample is prepared in said reaction chamber.

31. The method as described in claim 30 wherein said reagent vessels comprise a tubular member, a stopper member and a seal member, and wherein said step of sequentially subjecting said specimen in said reaction chamber to reagents from said reagent vessels comprises forcing said stopper member in each of said reagent vessels down said tubular member and opening said seal member and thereby dispensing said reagent in said reagent vessel into said reaction chamber.

32. The method as described in claim 30 wherein said testing device further comprises a first pneumatic port in communication with said first waste reservoir, and wherein said step of exhausting waste materials into said first waste reservoir comprises pulling a vacuum at said first pneumatic port.

33. The method as described in claim 30 further comprising the step of dispensing said extracted and purified nucleic acid sample into a PCR device, wherein said sample is amplified.

34. The method as described in claim 33 wherein said testing device further comprises a second pneumatic port and a second waste reservoir, and wherein said step of dispensing said extracted and purified nucleic acid sample into a PCR device comprises pulling a vacuum at said second pneumatic port.

35. The method as described in claim 30 further comprising the step of dispensing magnetic beads into said reaction chamber.

36. The method as described in claim 35 further comprising the step of magnetically retaining said magnetic beads in said reaction chamber when waste reagents are being exhausted.

37. A method of extracting, purifying and amplifying a nucleic acid sample, said method comprising the steps of:
collecting a nucleic acid sample;
inserting said sample into a testing device comprising an upper portion, a middle portion and a lower portion, said upper portion comprising a first rotatable housing member having a plurality of reagent vessels for storing reagents, a channel for storage of a collection dropper member, a collection dropper member, and a gear wheel for rotating said upper portion;
said middle portion comprising a second housing member having a reaction chamber, two pneumatic ports, a first waste reservoir and a second waste reservoir;
said lower portion comprising a base member and a PCR device;
at least one heat conducting mechanism for applying heat to said PCR device; and
a vacuum mechanism for applying a vacuum to said two pneumatic ports. sequentially subjecting said sample in said reaction chamber to reagents from said reagent vessels;
exhausting waste reaction materials in said reaction chamber to said first waste reservoir after each sequential reagent reaction;
wherein an extracted and purified nucleic acid sample is prepared in said reaction vessel;
subsequently dispensing said extracted and purified nucleic acid sample in said reaction chamber to said PCR device in said lower portion; and
subjecting said nucleic acid sample in said PCR device to denaturing, annealing and extension steps;
wherein the amount of said extracted and purified sample is amplified.

38. The method as described in claim 37 wherein said reagent vessels comprise a tubular member, a stopper member and a seal member, and wherein said step of sequentially subjecting said specimen in said reaction chamber to reagents from said reagent vessels comprises forcing said stopper member in each of said reagent vessels down said tubular member and opening said seal member and thereby dispensing said reagent in said reagent vessel into said reaction chamber.

39. The method as described in claim 37 wherein said testing device further comprises a first pneumatic port in communication with said first waste reservoir, and wherein said step of exhausting waste materials into said first waste reservoir comprises pulling a vacuum at said first pneumatic port.

40. The method as described in claim 37 further comprising a detection mechanism and further comprising the step of detecting the amount of sample being amplified in said PCR device.

41. The method as described in claim 40 wherein said detection mechanism is a fluorescent mechanism.

42. The method as described in claim 37 further comprising a magnetic mechanism positioned adjacent said reaction vessel.

43. The method as described in claim 42 wherein said magnetic mechanism is an electromagnetic mechanism.

44. The method as described in claim 37 wherein said reaction chamber has two outlet ports, a first port in communication with said first waste reservoir and a second port in communication with said PCR device and said second waste reservoir.

45. The method as described in claim 37 wherein the first of said two pneumatic ports is in communication with said first waste reservoir and the second of said two pneumatic ports is in communication with said second waste reservoir.

46. The method as described in claim 37 wherein said upper portion and said middle portion are rotatably connected together by a pin member.

47. The method as described in claim 37 further comprising a pair of post members on said base member of said lower portion, said pair of post members connecting said lower portion to said middle portion.
